(19) United States
(12) Patent Application Publication
(10) Pub. No.: US 2003/0069203 A1
(43) Pub. Date: Apr. 10, 2003

(54) Method for increasing human performance by reducing muscle fatigue and recovery time through oral administration of adenosine triphosphate

(76) Inventors: Steve S. Lee, Sandy, UT (US); Richard B. Hynson, Missoula, MT (US); Joe Zhou, (US)

Correspondence Address: PATE PIERCE & BAIRD, 215 SOUTH STATE STREET, SUITE 550, PARKSIDE TOWER, SALT LAKE CITY, UT, 84111, US

(21) Appl. No.: 10/162,143

(22) Filed: Jun. 3, 2002

Related U.S. Application Data
(60) Provisional application No. 60/295,705, filed on Jun. 4, 2001.

(51) Int. Cl. A61K 31/7076

(52) U.S. Cl. 514/47

(57) ABSTRACT

Systems and methods for delivering oral administration of ATP in a manner that protects the ATP from degradation by gastric juices through enteric coating to enhance absorption into the blood stream and provide additional therapeutic benefit when compared with non-protected forms of ATP. Said systems and methods comprising a composition used for improving muscle torque and reducing muscle fatigue, said composition comprising an effective amount of ATP. Preferably, a gastric acid secretion inhibitory coating is applied to the effective amount of ATP in a manner that protects the ATP from degradation by gastric juices. Said systems and methods effecting intracellular and extracellular ATP concentrations and increasing human performance by reducing muscle fatigue and recovery time which comprises administering an effective amount of ATP to a human. Alternatively, an effective amount of ATP may be administered sublingually, thereby avoiding exposure to gastric juices. The effective amount of ATP may be delivered by means of a tablet, granules, microgranules or powders.

### BACKGROUND

1. Related Application

This application claims the benefit of U.S. Provisional Application Serial No. 60/295,705, filed Jun. 4, 2001, and entitled “METHOD FOR INCREASING HUMAN PERFORMANCE BY REDUCING MUSCLE” which is hereby incorporated herein by reference.

2. Field of the Invention

This invention relates to the use of Adenosine Triphosphate (“ATP”) and, more particularly, to novel systems and methods for oral administration of ATP as a dietary supplement for the enhancement of human performance by increasing endurance and work capacity through reduction in muscle fatigue and decrease in muscle recovery time after exhaustion.

3. The Background

The biological importance of ATP first became apparent with the discovery of ATP in muscle tissue infusions by Fiske and Lohmann et al. in 1929. A. Szent-Gyorgi took the next logical step by demonstrating that ATP played an important role in muscle contraction. His experiments involved the addition of ATP to muscle fibers and then observing the subsequent contractions. Various researchers and those skilled in the art have progressively elucidated the role of ATP in muscle function since then. From these beginnings came the understanding and appreciation that ATP is the essential energy production molecule for every cell in the body. Similar phosphate-rich compounds are also found in every organism with ATP related compounds supplying all cellular energy. In 1982, Chaudry at the Yale Medical School published results showing that ATP was present in intracellular and interstitial fluids, thereby suggesting ATP's greatly expanded biological importance.

ATP and its breakdown product adenosine are also inherently involved in a number of extracellular processes like that of muscle contraction as described above. For example, some of these extracellular processes include neurotransmission, cardiac function, platelet function, vasodilatation and liver glycogen metabolism. As can be appreciated, these additional biological roles have given rise to various clinical applications of ATP and adenosine. For example, clinical applications may include applications of ATP and adenosine as a neuropathic and ischemic anesthetic, a hypotensive agent for trauma or disease induced hypertension such as pulmonary hypertension, a mild hypoglycemic in type II diabetes and at least preliminary evidence that ATP may be useful as an adjunctive therapy for radiation cancer treatment.

ATP and related compounds have been researched extensively for possible drug uses (see Daly, J. Med. Chem., 25:197, (1982)). The most widespread of these applications is in various cardiac treatments including the prevention of reperfusion injury after cardiac ischemia or stroke, and treatment of hypertension (see Jacobson, et al., J. Med. Chem., 35, 407-422 (1992)) as well as the treatment of paroxysmal supra ventricular tachycardia (see Pantely, et al., Circulation, 82, 1854 (1990)).

With regards to human performance specifically, the splitting of ATP to form adenosine diphosphate (ADP) is of critical importance in the functioning of muscle, since this is the reaction that directly supplies energy to myosin and actin to facilitate normal muscular contraction. In many cases, this requirement is met by the actual rebuilding of ATP as it is used, rather than by storing a very large amount of ATP in the muscle. However, under exceptionally demanding conditions such as peak athletic performance or certain deficiency states induced by either inadequate nutrition or various diseases, ATP availability could prove to be a limiting step in actuating peak muscle output.

While therapeutic uses of ATP in various disease states is quite common, applications of ATP relating to possible benefits such as increased athletic performance in normal, healthy individuals appear to be largely absent in the published literature.

A prior art method of increasing intracellular ATP through orally administered precursors of adenosine triphosphate in dietary supplements for treatment of reduced energy availability resulting from strenuous physical activity, illness or trauma is disclosed in U.S. Pat. No. 6,159,942. However, ATP itself is not administered; rather pentose sugars are administered individually, mixed into dry food or in solution. Specifically, the preferred pentose is D-ribose, singly or combined with creatine, pyruvate, L-camitine and/or vasodilating agents.

As appreciated by those skilled in the art, the mechanism of action for ribose to stimulate ATP production is through the phosphorylation of nucleotide precursors that may be present in the tissues. These are converted to adenosine monophosphate (AMP) and further phosphorylated to ATP. Adenosine is directly phosphorylated to AMP, while xanthine and inosine are first ribosylated by 5-phosphoribosyl-1-pyrophosphate (PRPP) and then converted to AMP. In the de novo synthetic pathway, ribose is phosphorylated to PRPP, and condensed with adenine to form the intermediate AMP. AMP is further phosphorylated via high energy bonds to form adenosine diphosphate (ADP) and ATP.

In certain circumstances, ATP can cross directly into the cell without the need for intracellular de novo synthesis. Chaudry (1982) explained that exogenous ATP crosses cellular membranes when depletion occurs within myosin units. ATP or ATP substrates may access human physiology orally, sublingually or intravenously. Carbohydrates, oral ATP or oral-sublingual ATP may be consumed for enhancing endurance performance, and preventing muscle exertion or heat stress cramps. Therefore, methods of delivering actual ATP to the bloodstream and subsequently to interstitial fluids may have benefits not associated with mere ATP precursors.

In addition to exhibiting the proper therapeutic effect, any method for delivering actual ATP to muscle cells in an attempt to prevent depletion must also include a consideration of the realities of the practical administration of a therapeutic agent in a daily athletic environment. First, the therapeutic agent must be suitable for sale as a dietary supplement and not only as a drug. This requires that the therapeutic agent have certain technical and economic characteristics related to the dietary supplement industry. From a technical standpoint, the therapeutic agent should preferably be orally administered and suitable for inclusion in a variety of dosage forms such as tablets or capsules or included in solid foods mixed into dry food or in solution. Additionally, the therapeutic agent should also be well tolerated vis a vis digestion and be suitably stable both ex vivo and in vivo.

From an economic standpoint, a therapeutic agent should ideally be robust enough for combination with a variety of other ingredients without the need for special handling during manufacture or special processing, packaging or storing of the resulting composition or mixture.

ATP is generally known to be subject to degradation from exposure to high temperature and/or high humidity conditions and in the presence of a low pH such as that found in stomach acid. It is therefore desirable to protect parenterally administered ATP from degradation by stomach acid through the use of a low pH insoluble compound, such as a protective enteric coating. Sublingual ATP preparations, which are not subject to exposure to gastric fluids, exist but they are not suitable for inclusion in a variety of dosage forms and complex formulations. This creates the need to coat supplements containing currently available ATP (such as adenosine-5′-triphosphate disodium) to impart protective enteric properties after the final dosage form is manufactured.

While the technique of enteric coating has been applied to finished ATP dosage forms such as capsules and tablets, it has not been applied to granular ATP preparations suitable for inclusion in alternate dosage forms common to nutritional supplements such as liquids, nutrition bars and powders, as well as, the above-mentioned tablets and capsules.

Consistent with the foregoing, an ideal ATP preparation should include protective enteric properties independent of the final dosage form, thus eliminating the need for potential customers to impart enteric protection during manufacture since this capability is both expensive and uncommon. Additionally, providing enteric protection for finished food dosage forms such as liquids, bars and powders is not possible.

### SUMMARY AND OBJECTS OF THE INVENTION

In view of the foregoing, it is a primary object of the present invention to provide novel systems and methods for providing an increased supply of ATP and the subsequent ADP generation that is demonstrably the limiting step in optimal muscle output, endurance and recovery.

It is a further object of the present invention to provide novel systems and methods for delivering oral administration of ATP in a manner that protects the ATP from degradation by gastric juices through enteric coating to enhance absorption into the blood stream and provide additional therapeutic benefit when compared with non-protected forms of ATP.

It is also an object of the present invention to provide novel systems and methods for coating ATP enterically that are compatible with manufacture of foods, drugs and dietary supplements of complex formulation and various dosage forms including capsules, tablets, caplets, lozenges, liquids, sublingual, solid foods, powders and other conceivable dosage forms, as applicable, without the need for imparting enteric properties to the entire mixture, any other part of the mixture, or finished products.

Consistent with the foregoing objects, the present invention provides systems and methods for delivering oral administration of ATP in a manner that protects the ATP from degradation by gastric juices through enteric coating to enhance absorption into the blood stream and provide additional therapeutic benefit when compared with non-protected forms of ATP. Said systems and methods comprising a composition used for improving muscle torque and reducing muscle fatigue, said composition comprising an effective amount of ATP. Preferably, a gastric acid secretion inhibitory coating is applied to the effective amount of ATP in a manner that protects the ATP from degradation by gastric juices. As contemplated herein, the effective amount of ATP may be delivered by means of a tablet, granules, microgranules or powders.

### DETAILED DESCRIPTION OF THE PREFERRED EMBODIMENTS

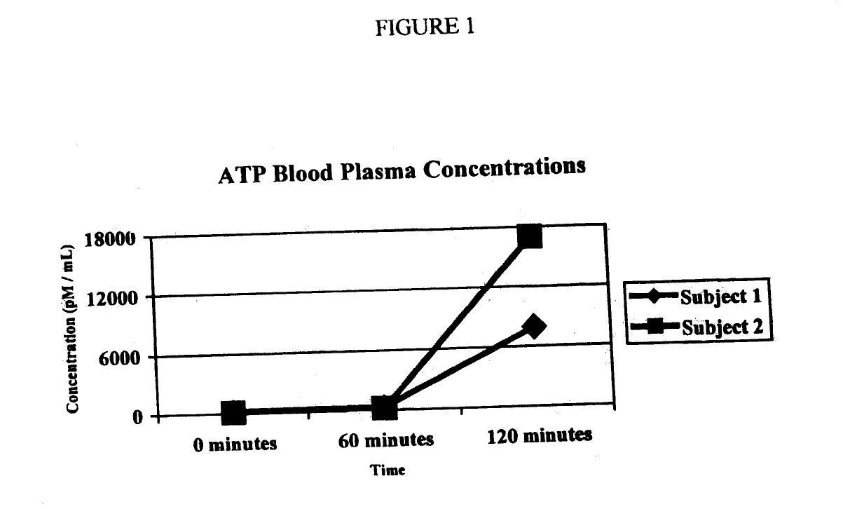

It will be readily understood that the components of the present invention, as generally described and illustrated in the Figures herein, could be modified, arranged and designed in a wide variety of different configurations. Thus, the following more detailed description of the embodiments of the systems and methods of the present invention, as represented in the Examples and FIGS. 1 through 10, is not intended to limit the scope of the invention. The scope of the invention is as broad as claimed herein.

Oral administration of ATP is usually in the form of Adenosine-5′-Triphosphate Disodium. For the purpose of contemplating the breadth and scope of the present invention, Adenosine-5′-Triphosphate Disodium or any form of ATP or adenosine suitable for oral administration may be combined with any of the known coatings suitable for imparting enteric properties in granular form.

Granular formation or agglomeration may be achieved by means of any conventional method including for example fluidized bed granulation, wet granulation or spherical rotation agglomeration. Subsequent enteric coatings include, for example but not by way of limitation, methacrylic acid-acrylic acid copolymers, cellulose acetate phthalate, hydroxypropylmethylcellulose phthalate and acetate succinate, shellac, polyethylene glycol, polysorbates, carboxymethylcellulose or polyoxyethylene-polyoxypropylene glycol. Furthermore, the objects of the present invention may be at least partially accomplished through the use of quasi-enteric coatings or materials such as those which result in delayed or timed release of active ingredients such as sugars, castor oil, microcrystalline cellulose, starches such as maltodextrin or cyclodextrin, or food-grade gums or resins.

A water barrier overcoat may then be applied to assist in isolating the ATP active from other formulation ingredients as well as provide protection versus environmental degradation.

In human performance enhancing formulations, the resulting ATP granules would be incorporated in a fashion so as to result in a typical per dose dosage range of 25 mg to 600 mg, though more or less may be desirable depending on the application and other ingredients. In one presently preferred embodiment of the present invention, this dosage range may be administered two (2) to three (3) times per day for maximum effectiveness.

The following examples will illustrate the invention in further detail. It will be readily understood that the composition of the present invention, as generally described and illustrated in the Examples herein, could be synthesized in a variety of formulations and dosage forms. Thus, the following more detailed description of the presently preferred embodiments of the methods, formulations, and compositions of the present invention, as represented in Example I is not intended to limit the scope of the invention, as claimed, but it is merely representative of the presently preferred embodiments of the invention.

### EXAMPLE I

21 mg of Adenosine-5′-Triphosphate Disodium was entabletted in a Stokes B2, 16 station tablet press using ⅜″ standard concave punch dies. Tablets included microcrystalline cellulose as an inert filler and less than 3% magnesium stearate as a lubricant. Total tablet weight was 350 mg. Resulting tablet hardness was approximately 12 kp. The tablet cores were then coated with ten percent methacrylic copolymer (Eudragit from Rohm, West Germany).

The tablets were then given to two (2) healthy male volunteers, ages 51 and 57, respectively, for the purpose of evaluating the ability of the present invention to deliver ATP to blood plasma. FIG. 1 shows the increase in ATP blood plasma levels following administration.

As these results clearly show, the present invention results in dramatically increased ATP blood plasma concentrations in a manner consistent with effective enteric delivery.

### EXAMPLE II

25 mg of Adenosine-5′-Triphosphate Disodium was entabletted in a Stokes B2, 16 station tablet press using ⅜″ standard concave punch dies. Tablets included microcrystalline cellulose as an inert filler and less than 3% magnesium stearate as a lubricant. Total tablet weight was 350 mg. Resulting tablet hardness was approximately 12 kp. The tablet cores were then coated with ten percent (10%) methacrylic copolymer. (See Eudragit from Rohm, West Germany.)

The tablets were then given to twenty-one volunteers for the purpose of evaluating the effectiveness of the present invention as an aid to enhancing human performance:

| | | | | Number |
|---|---|---|---|---|
| | | Avg Weight (kg) | Age (years) | in Group (n) |
| Control: | Males | 84.5 | 26.1 | 6 |
| | Females | 63.1 | 30.7 | 4 |
| ATP: | Males | 76.1 | 28.0 | 7 |
| | Females | 58.0 | 22.4 | 4 |

Doses were given in double blind fashion with neither the recipient nor the researcher aware of active versus placebo administration. Results were measured using a standard Wingate test for measuring endurance. Since the 1970's the Wingate test has become “one of the most widely recognized protocols in exercise research for determining peak muscle power and indirectly reflecting anaerobic capacity.” (Roberg and Roberg, Exercise Physiology, Musky Publishers 1997) The test consists of pedaling or arm cranking at maximal effort for thirty seconds against a constant load.

The experiment specifically sought to measure muscle recovery following the administration of a single Wingate maximal effort test lasting 15 seconds by contrasting the output with a second Wingate maximal effort test conducted immediately following the first test. The results were measured for a period of 120 minutes with the first pair of tests conducted beginning two hours after administration of the present invention and then again every 30 minutes thereafter.

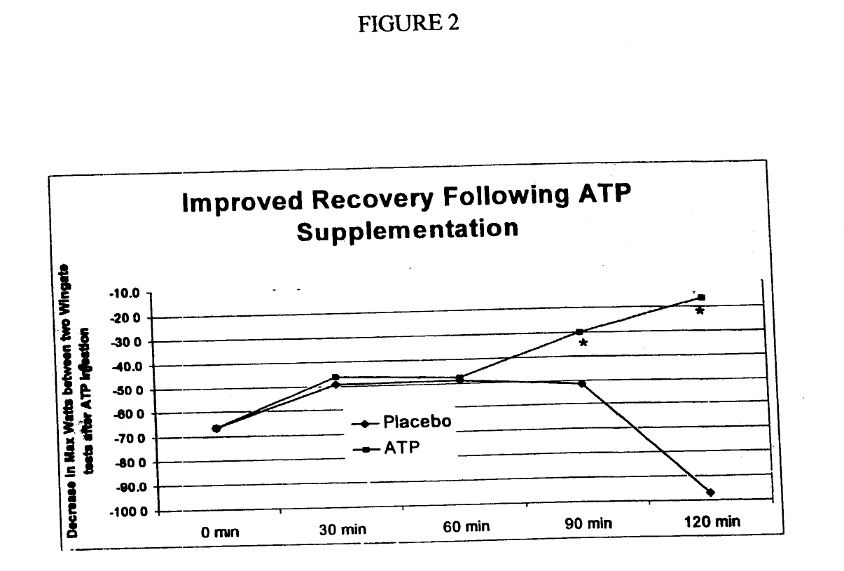

FIG. 2 illustrates the results of the experiment:

The results show substantially improved muscle recovery and substantially less depletion of maximal output versus placebo following administration of the dosage of ATP. The results also indicate a persistent effect that peaks sometime around or after 120 minutes.

### EXAMPLE III

Using the same tablet preparation as in Example II, another series of tests was conducted to evaluate the effects of a single dose containing about 25 mg ATP on various parameters measuring performance using three back-to-back Wingate tests . The first test was administered two (2) hours after oral administration of the invention. The following Figures illustrate several different measurements of this series of tests.

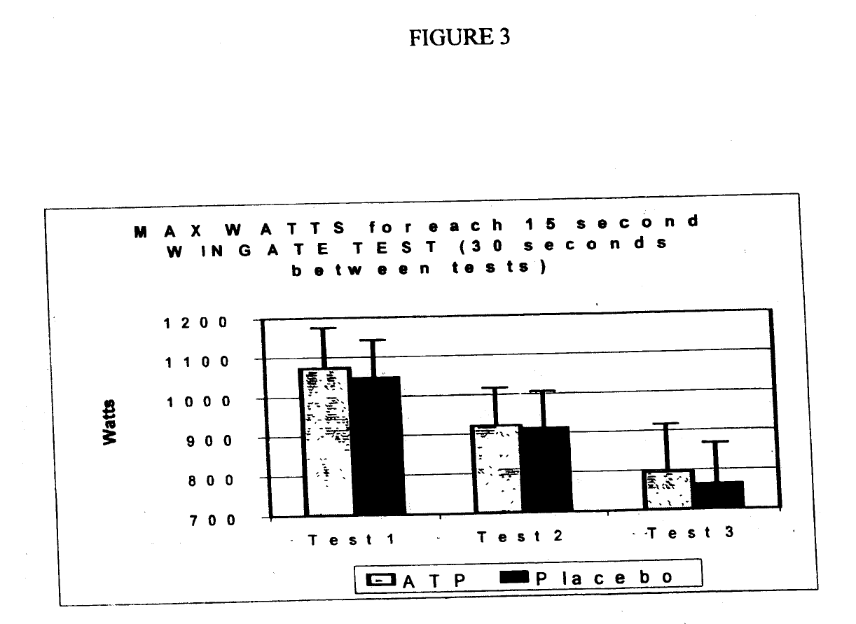

FIG. 3 shows the level of maximum muscle output during the entire 15-second test for each of the three back-to-back tests following administration versus placebo.

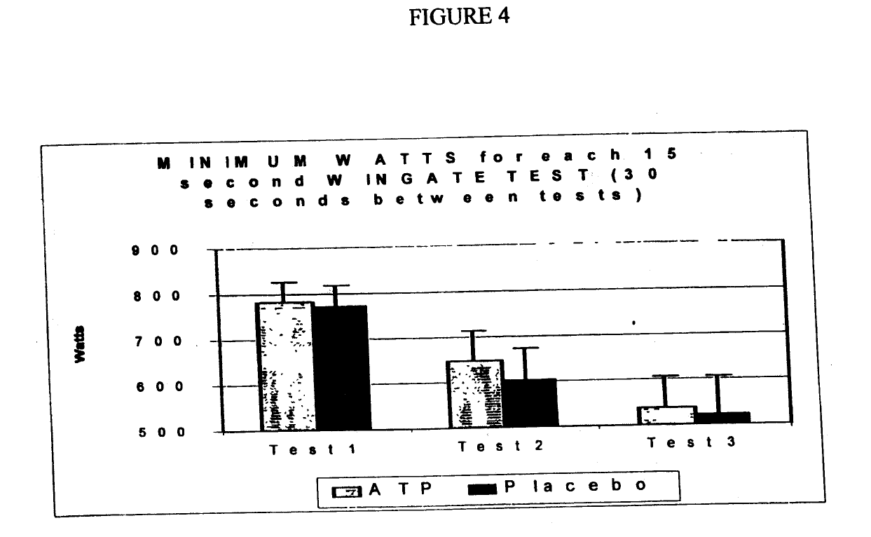

FIG. 4 shows the level of minimum muscle output during the entire 15-second test for each of the three back-to-back tests following administration versus placebo.

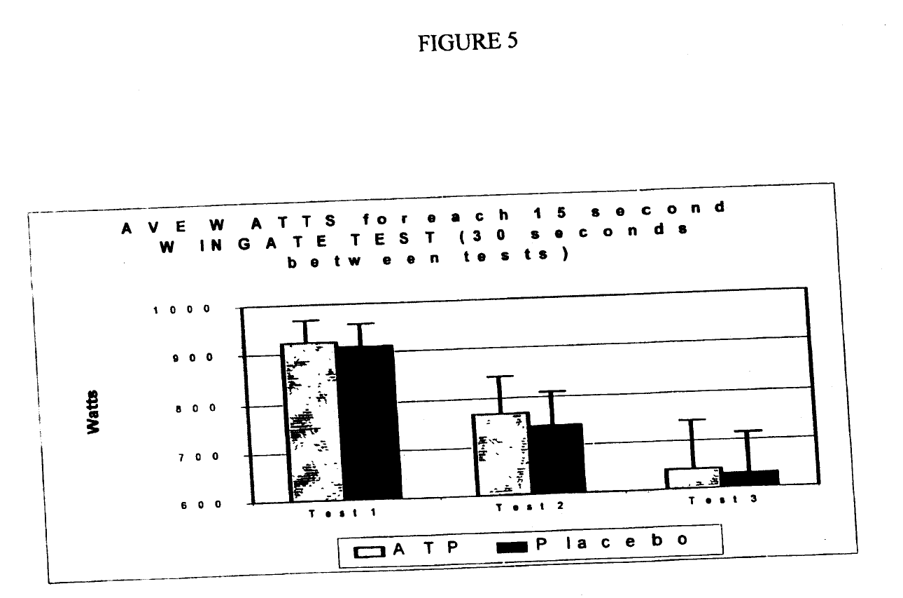

FIG. 5 shows the level of average muscle output during the entire 15-second test for each of the three back-to-back tests following administration versus placebo.

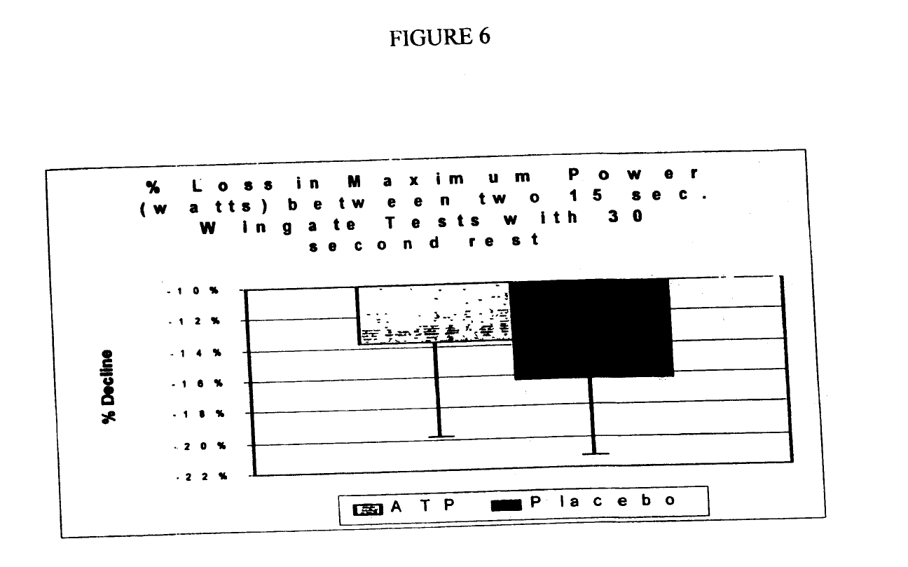

FIG. 6 shows the decrease in maximum muscle output between the first and second Wingate test following administration versus placebo.

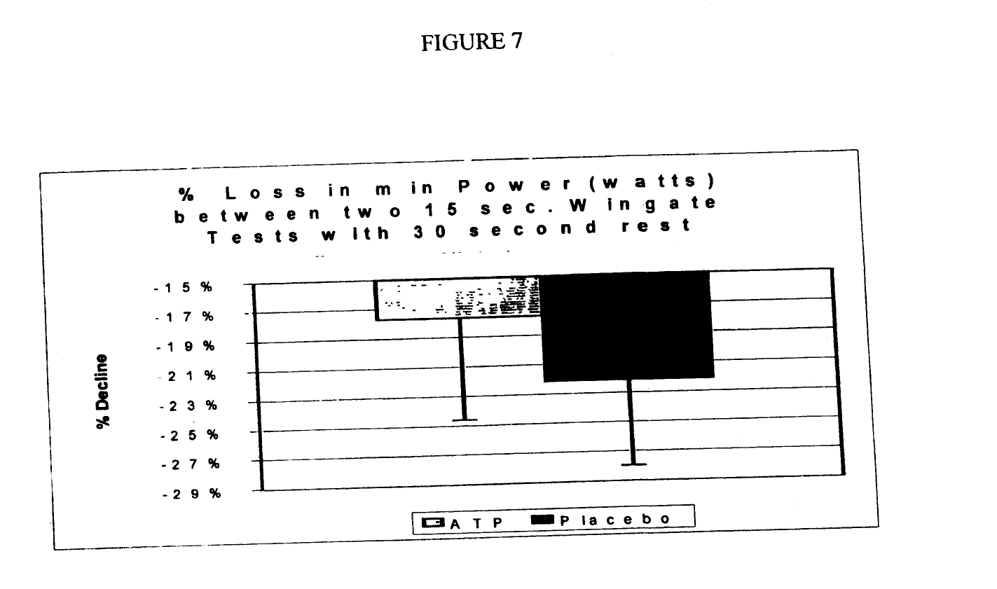

FIG. 7 shows the decrease in minimum muscle output between the first and second Wingate test following administration versus placebo.

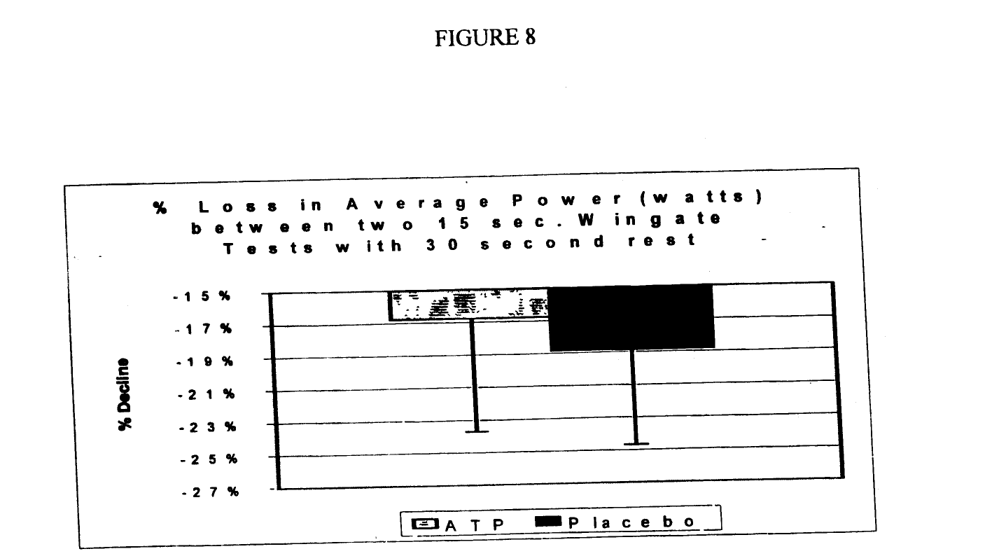

FIG. 8 shows the decrease in average muscle output between the first and second Wingate test following administration versus placebo.

Adenosine-5′-Triphosphate Disodium was agglomerated into granules using a seed crystal nucleus upon which a mixture containing ATP and various excipients for binding and flow was progressively loaded using a fluidized bed processor. In one presently preferred embodiment of the present invention, the base granulation formula was approximately, as follows:

20% ATP

20% Microcrystalline Cellulose

20% Starch

35% Sucrose

5% Maltodextrin

The resulting agglomeration was then dried with a loss of weight on drying of about 1% to 4% yielding a granule from 100 to 1000 microns in size with an active ATP “drug” load of approximately 10% to 30%. The loaded particles were then coated with about 15% to 40% aqueous enteric coating containing sixty-three percent (63%) (Emcoat 120N), nineteen and one-half percent (19.5%) Hydroxypropylmethylcellulose (HPMC), twelve and one-half percent (12.5%) Oleic acid and five percent (5%) Triacetin. The prepared granules were encapsulated in two-piece hard gelatin capsules using microcrystalline cellulose as a filler and less than 3% magnesium stearate as a lubricant.

### EXAMPLE V

Using the same tablet preparation as in Examples II and III, another test was conducted to evaluate the bioavailability of a single dose containing about 850 mg ATP. The tablets were given to two volunteers for the purpose of evaluating relative changes in intracellular and extracellular ATP levels following the dosage. The dosage was administered on an empty stomach; volunteers fasted from midnight until the test, about 8 hours later. One volunteer received a dose about 15 mg active ATP/kg and the second volunteer received a dose about 7.5 mg active ATP/kg.

A baseline blood ATP level was obtained immediately prior to dosage administration and additional ATP blood levels were obtained at intervals of 30 minutes, 1 hour, 2 hours, 4 hours, and 6 hours following dosage administration.

The following Figures illustrate the results from this test.

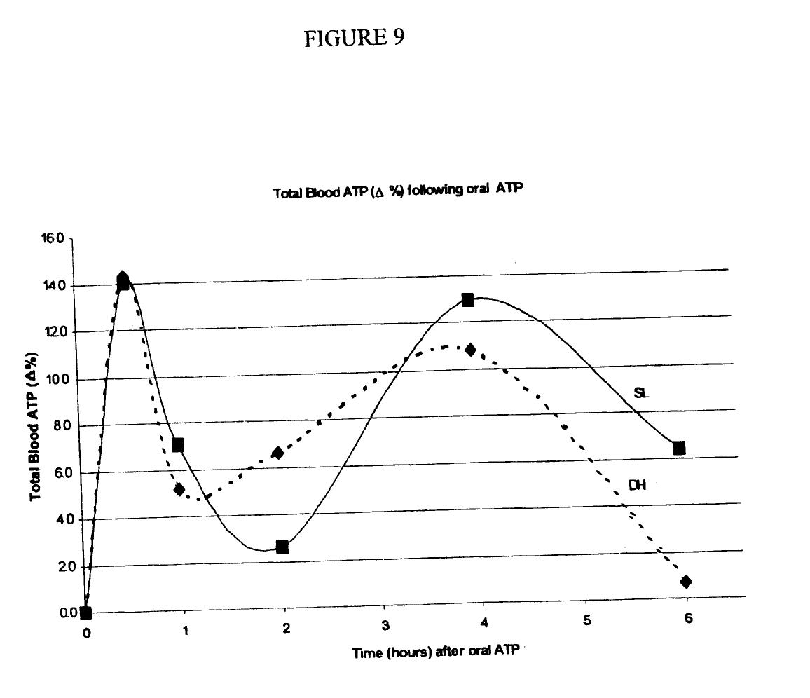

FIG. 9 shows the percentage change of the concentration of ATP in total blood over 6 hours following dosage administration.

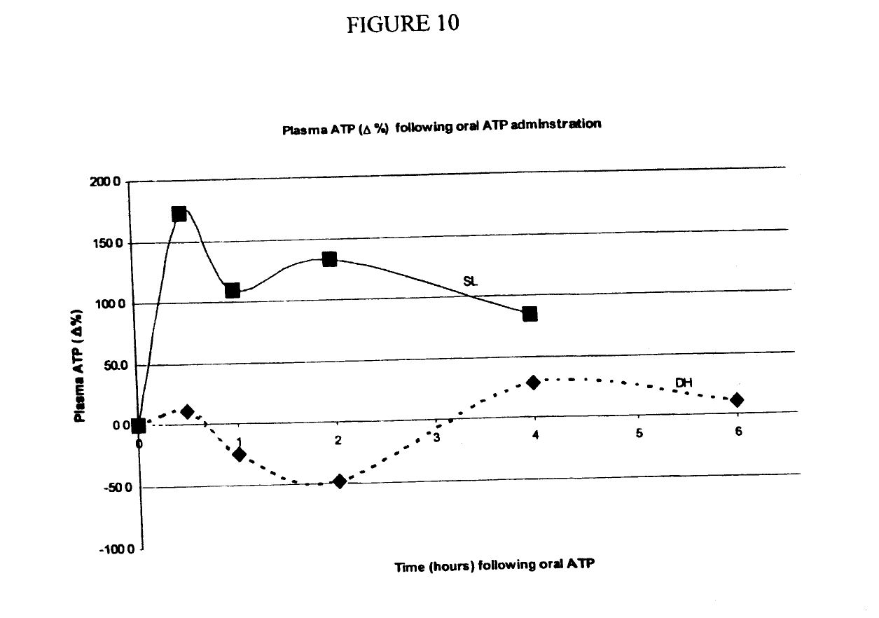

FIG. 10 shows the percentage change of the concentration of ATP in plasma over 6 hours following dosage administration.

All results represented in FIGS. 1 through 10 are statistically accurate.

The experiment set forth in Example V specifically sought to measure the presence of a pharmacokinetic dose-response within the intracellular and extracellular body compartments following the administration of a single dosage of the present invention.

Based on the foregoing findings, FIGS. 9 and 10 demonstrate that there is a measurable relationship between the oral administration of an effective amount of ATP and alterations in blood and plasma concentrations of ATP. Moreover, FIGS. 1 through 8 demonstrate a measurable relationship between the oral administration of an effective amount of ATP and human physical performance testing. These data show that the present invention provides a method for effecting intracellular and extracellular ATP concentrations and increasing human performance by reducing muscle fatigue and recovery time which comprises administering an effective amount of ATP to a human in need of such treatment.

As these examples demonstrate, the present invention provides a method for effecting intracellular and extracellular ATP concentrations in mammals. Additionally, the present invention substantially increases human performance by increasing endurance and muscle output through reduction in muscle fatigue and decrease in muscle recovery time after exhaustion. Moreover, the present invention provides systems and methods for delivering oral administration of ATP in a manner that protects it from degradation by gastric juices through enteric coating to enhance absorption into the blood stream or through avoiding exposure to gastric juices by sublingual administration, and provide additional therapeutic benefit when compared with non-protected forms.

The foregoing examples outlined herein also illustrate systems and methods for enterically coating ATP compatible with manufacture of foods, drugs and dietary supplements of complex formulation and various dosage forms without the need for imparting enteric properties to the entire mixture, any other part of the mixture, or finished products.

The present invention may be embodied in other specific forms without departing from its structures, methods, or other essential characteristics as broadly described herein and claimed hereinafter. The described embodiments are to be considered in all respects only as illustrative, and not restrictive.

The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A composition comprising Adenosine Triphosphate (“ATP”) in an amount effective for improving muscle torque and reducing muscle fatigue in mammals.

2. The composition as defined in claim 1, wherein said amount of ATP is orally administered.

3. The composition as defined in claim 2, wherein said amount of ATP is in a tablet form.

4. The composition as defined in claim 2, wherein said amount of ATP is in a granular form.

5. The composition as defined in claim 4, wherein said granular form comprises microgranules.

6. The composition as defined in claim 2, wherein said amount of ATP is in a powder form.

7. The composition as defined in claim 2, wherein a gastric acid secretion inhibitory coating is applied to said amount of ATP.

8. The composition as defined in claim 7, wherein said gastric acid secretion inhibitory coating is comprised of a methacrylic copolymer.

9. The composition as defined in claim 7, wherein said inhibitory coating comprises a range of 1 to 20% w/w.

10. The composition as defined in claim 7, wherein said inhibitory coating comprises 10% w/w.

11. The composition of claim 1, wherein said amount of ATP is parentally administered.

12. A composition used for effecting intracellular and extracellular concentrations of ATP, said composition comprising an effective amount of ATP and a gastric acid secretion inhibitory coating applied to said amount of ATP.

13. The composition as defined in claim 12, wherein said gastric acid secretion inhibitory coating comprises a methacrylic copolymer.

14. The composition as defined in claim 12, wherein said amount of ATP comprises a tablet form.

15. The composition as defined in claim 12, wherein said amount of ATP comprises a granular form.

16. The composition as defined in claim 12, wherein said amount of ATP comprises a microgranular form.

17. The composition as defined in claim 12, wherein said amount of ATP comprises a powder form.

18. A method for administering an effective amount of ATP for effecting intracellular and extracellular ATP concentrations and increasing human performance by reducing muscle fatigue and recovery time.

19. The method as defined in claim 18, further comprising the step of applying a gastric acid secretion inhibitory coating to said ATP.

20. The method of claim 19, wherein, said inhibitory coating comprises a range of about 1% w/w to 20% w/w.
